(19) United States
(12) Patent Application Publication
(10) Pub. No.: US 2025/0185088 A1
(43) Pub. Date: Jun. 5, 2025

(54) PLATFORM FOR CONNECTING PERIPHERAL DEVICES TO EXERCISE MACHINES

(71) Applicant: Peloton Interactive, Inc., New York, NY (US)

(72) Inventors: Eric KISS, Brooklyn, NY (US); Daniel HERTZ, New York, NY (US); Mottaqui KARIM, South Orange, NJ (US); Kyle E. JABLONSKI, West Chester, PA (US); Dalton Lee CLAYBROOK, Brooklyn, NY (US); Nicholas BREESER, New York, NY (US); Jackson MASLOW, Brooklyn, NY (US); Dean Rex SILFEN, New York, NY (US); Katie MCNABB, Brooklyn, NY (US)

(21) Appl. No.: 18/839,268

(22) PCT Filed: Feb. 28, 2023
(86) PCT No.: PCT/US2023/014041

§ 371 Date: Aug. 16, 2024

Related U.S. Application Data
(60) Provisional application No. 63/314,676, filed on Feb. 28, 2022.

(51) Int. Cl. H04W 76/14; A63B 24/00

(52) U.S. Cl. CPC H04W 76/14; A63B 24/0062; A63B 2225/20; A63B 2225/50

(57) ABSTRACT

Various systems and methods that support or facilitate the exchange of data between devices within a connected fitness networked environment are described. In some embodiments, the systems and methods include a remote or cloud-based data service, which connects various devices to one another, such as peripheral devices to exercise machines within the networked environment.

### BACKGROUND

This application claims priority to U.S. Provisional Patent Application No. 63/314,676, filed on Feb. 28, 2022, entitled PLATFORM FOR CONNECTING PERIPHERAL DEVICES TO EXERCISE MACHINES, which is hereby incorporated by reference in its entirety.

### BACKGROUND

The world of connected fitness is an ever-expanding one. This world can include a user taking part in an activity (e.g., running, cycling, lifting weights, and so on), other users also performing the activity, and many other users doing other activities. The users may be exercising on a fitness machine (e.g., a treadmill, a stationary bike, a strength machine, a stationary rower, and so on), or may be moving through the world on a bicycle, boat, skiing, or other exercise equipment.

The users can also be performing activities that do not include an associated machine or other equipment, such as running, strength training, yoga, stretching, hiking, climbing, and so on. These users can have a wearable device or mobile device that monitors the activity and may perform the activity in front of a user interface (e.g., a display or device) presenting content associated with the activity.

The user interface, whether a mobile device, a display device, or a display that is part of a machine, can provide or present interactive content to the users. For example, the user interface can present live or recorded classes, video tutorials of activities, leaderboards and other competitive or interactive features, progress indicators (e.g., via time, distance, and other metrics), virtual games or interactive experiences, augmented or mixed reality scenarios, and so on.

For example, the interactive content can include video or images that mimic or simulate the user traveling or moving (e.g., running, biking, rowing, and so on) through a virtual environment, such as a virtual game or other digitally rendered interactive environment.

In some cases, the user wears a peripheral device or accessory, such as a heart rate monitor (HRM), smart watch, or other wearable device, during an exercise activity. The peripheral device can measure data or information about the user, such as their heart rate, their temperature, and so on. Further, the peripheral device, in some cases, can display information to the user and/or facilitate the input of requests from the user, such as via a user interface or touchscreen of the device.

### BRIEF DESCRIPTION OF THE DRAWINGS

Embodiments of the present technology will be described and explained through the use of the accompanying drawings.

FIG. 1 is a block diagram illustrating a suitable network environment for connecting a peripheral device to an exercise machine within a connected fitness platform.

FIG. 2 is a block diagram illustrating a data flow architecture for exchanging data between a peripheral device and an exercise machine.

FIG. 3 is a block diagram illustrating a data flow architecture that provide a backward capability for a peripheral device communicating with an exercise machine.

FIG. 4 is a flow diagram illustrating a method for transmitting data between a peripheral device and an exercise machine.

FIG. 5 is a data flow diagram illustrating the flow of data within a cloud-based metrics service.

FIG. 6 is a data flow diagram illustrating the flow of data within a cloud-based metrics service having a separate metrics service.

FIG. 7 is a flow diagram illustrating a method for controlling an exercise machine with a peripheral device via a cloud-based metrics service.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

Various systems and methods that support or facilitate the exchange of data between devices within a connected fitness networked environment are described. In some embodiments, the systems and methods include a remote or cloud-based data service, which connects various devices to one another, such as peripheral devices to exercise machines within the networked environment.

For example, the systems and methods can facilitate the communication of metrics and other data between devices via the remote data service by determining whether a peripheral device can establish a direct connection with an exercise machine, and when the peripheral device cannot establish the direct connection with the exercise machine, establish a connection with a remote data service associated with the exercise machine and exchange data captured from a user of the exercise machine with the exercise machine via the established connection with the remote data service.

As another example, the systems and methods can utilize the data service as a metrics service for various services provided by a connected fitness platform, such as microservices. For example, the platform can include a metrics service that receives data packet from multiple exercise machines that contain data captured from users and/or the machines, and one or more microservices (e.g., a leaderboard service) that access the metrics service to update a service provided to the multiple exercise machines by the connected fitness platform.

Various embodiments of the systems and methods will now be described. The following description provides specific details for a thorough understanding and an enabling description of these embodiments. One skilled in the art will understand, however, that these embodiments may be practiced without many of these details. Additionally, some well-known structures or functions may not be shown or described in detail, so as to avoid unnecessarily obscuring the relevant description of the various embodiments. The terminology used in the description presented below is intended to be interpreted in its broadest reasonable manner, even though it is being used in conjunction with a detailed description of certain specific embodiments.

### Examples of a Suitable Network Environment

The technology described herein is directed, in some embodiments, to providing a data exchange mechanism and platform for connecting devices within a connected fitness platform or other exercise system. The systems and methods can utilize cloud-based services, such as remote or other cloud-based data services, when facilitating the exchange of information between devices, such as between peripheral devices (e.g., smart watches, heart rate monitors, other wearable devices, and so on) and exercise machines (e.g., treadmills, exercise bicycles, rowers, strength machines, and so on).

For example, some exercise machines require specific software to facilitate the exchange of information between the exercise machine and a peripheral device. The data exchange mechanism described herein seeks to enable the exchange of information without such software, such as via a remote data service that is configured to coordinate and/or manage the communications between the peripheral devices and exercise machines, among other benefits.

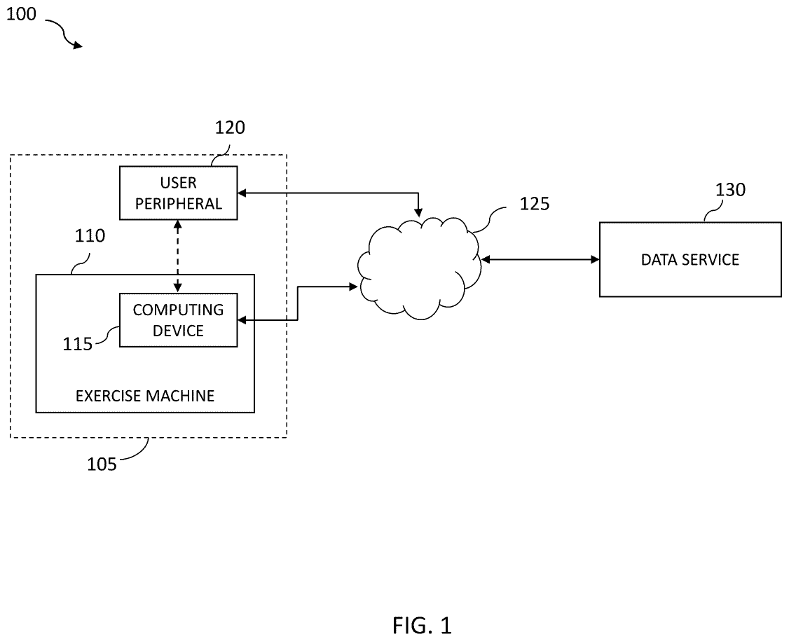

FIG. 1 is a block diagram illustrating a suitable network environment for connecting a peripheral device to an exercise machine within a connected fitness platform. The network environment 100 includes an activity environment 105, where a user is performing an exercise activity, such as a cycling activity. In some cases, the user can perform the activity with an exercise machine 110, such as cycling on an exercise bicycle, running on a treadmill, rowing on a rowing machine, lifting weights using a strength machine, and so on.

The exercise activity performed by the user can include a variety of different workouts, activities, actions, and/or movements, such as movements associated with stretching, doing yoga, lifting weights, rowing, running, cycling, jumping, sports movements (e.g., throwing a ball, pitching a ball, hitting, swinging a racket, swinging a golf club, kicking a ball, hitting a puck), and so on.

The exercise machine 110 can assist or facilitate the user to perform the movements and/or can present interactive content, such as live classes, pre-recorded classes, a digital game, and so on, to the user when the user performs the activity. For example, the exercise machine 110 can be a stationary bicycle, a stationary rower, a treadmill, a weight machine, or other machines. As another example, the exercise machine 110 can include a display device and associated computing device 115 that presents content (e.g., classes, dynamically changing video, audio, video games, instructional content, and so on) to the user during an activity or workout.

The computing device 115, which may be part of the display, can include or is associated with a media hub and/or a user interface. The media hub, in some cases, captures images and/or video of the user, such as images of the user performing different movements, or poses, during an activity. The media hub can include a camera or cameras, a camera sensor or sensors, or other optical sensors configured to capture the images or video of the user.

The user interface provides the user with an interactive experience, such as a class, digital video game or interactive game, guided tour of a virtual environment, virtual reality experience, augmented reality experience, and so on, during the activity. The exercise machine 110, via the computing device 115 or other communication components, can send or receive information over a network 125, such as a wireless network. Thus, in some cases, the user interface is a display device (e.g., attached to the exercise machine 110), that receives content from (and sends information, such as user selections) a server of a connected fitness platform, such as a data service 130, over the network 125.

In some cases, the user wears or is associated with a peripheral device 120, such as a smart watch, heart rate monitor (HRM), smart glasses, or other wearable devices, accessories, or performance trackers. The peripheral device 120 can capture or measure various types of information about the user or a performed exercise activity, such as metrics associated with a user's effort, movement, or biological responses to the activity (e.g., a heart rate).

Further, the peripheral device 120 can include similar components to the computing device 115, such as a user interface, communication components, and so on. The peripheral device 120 can present information associated with the exercise activity to the user via its own user interface, can receive user input via the user input, can directly communicate information to the computing device 115 of the exercise machine 110 (e.g., via a Bluetooth channel), and so on.

In some cases, the computing device 115 of the exercise machine 110 can include components or modules directly associated with the peripheral device 120, such as components or modules that facilitate the direct exchange of information between the exercise machine 110 and the peripheral device 120. For example, the computing device 115 can include a GymKit module, which directly synchronizes information between Apple provided peripheral devices (e.g., Apple watches) and the exercise machine 110. Thus, some peripheral devices can be associated with direct communication modules that are implemented into the computing device 115 of the exercise machine 110.

Therefore, the networked environment 100, in some embodiments, can support the exchange of information via direct communication channels and/or via the data service 140, which establishes communication channels over the network 125.

FIG. 1 and the components, systems, servers, and devices depicted herein provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, floppy diskettes, optical discs, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other types of media/machine-readable medium suitable for storing electronic instructions.

The network or cloud 125 can be any network, ranging from a wired or wireless local area network (LAN), to a wired or wireless wide area network (WAN), to the Internet or some other public or private network, to a cellular (e.g., 4G, LTE, or 5G network), and so on. While the connections between the various devices and the network 125 and are shown as separate connections, these connections can be any kind of local, wide area, wired, or wireless network, public or private.

Further, any or all components depicted in the Figures described herein can be supported and/or implemented via one or more computing systems, servers, or cloud-based systems. Although not required, aspects of the various components or systems are described in the general context of computer-executable instructions, such as routines executed by a general-purpose computer, e.g., mobile device, a server computer, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, microprocessor-based or programmable consumer electronics, set-top boxes, network PCs, mini-computers, mainframe computers, AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as an exercise machine, display device, or mobile or portable device, and thus, while certain hardware platforms are described herein, aspects of the system are equally applicable to nodes on a network. In some cases, the mobile device or portable device may represent the server portion, while the server may represent the client portion.

### Examples of a Peripheral Device Data Service for a Connected Fitness Platform

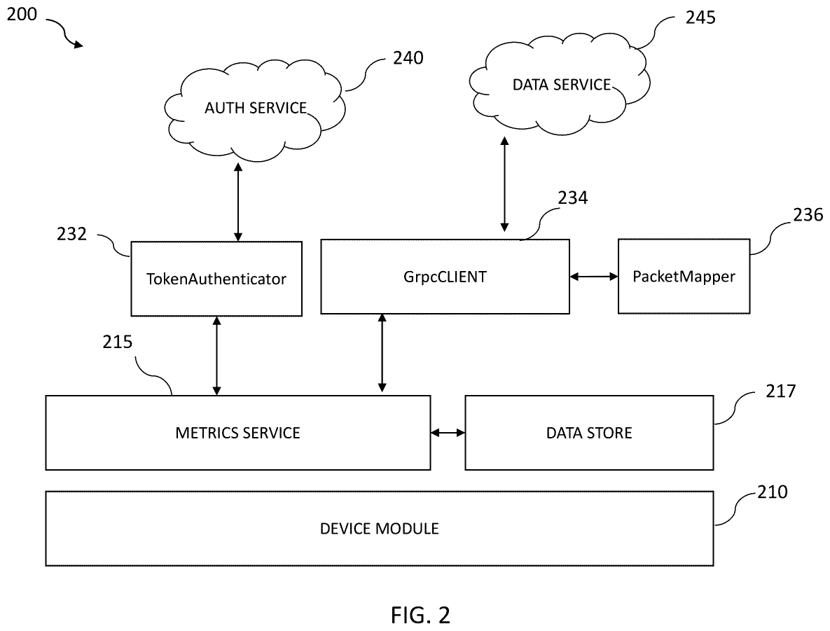

As described herein, in some embodiments, the systems and methods provide a data service to devices that measure data associated with exercise activities performed by users within a connected fitness platform, such as via exercise machines of the connected fitness platform. FIG. 2 is a block diagram illustrating a data flow architecture 200 for exchanging data between a peripheral device and an exercise machine.

The components and/or modules of the data flow architecture 200 (and other systems or architectures described herein) can be implemented with a combination of software (e.g., executable instructions, or computer code) and hardware (e.g., at least a memory and processor). Accordingly, as used herein, in some embodiments, a component/module is a processor-implemented component/module and represents a computing device having a processor that is at least temporarily configured and/or programmed by executable instructions stored in memory to perform one or more of the functions that are described herein.

The data flow architecture 200 includes a device module 210 that is stored, supported, and/or implemented by a peripheral device, such as the peripheral device 120. The device module 210 can be a mobile application downloaded to the peripheral device 120 and/or part of the device operating system, such as implemented as one or modules of the device OS.

A metrics service 215 and data store 217 are classes that are associated with the device module 210, such as via the mobile application that includes the device module 210. The metrics service 215 delegates requests, responses, or other messages from the device module 210, and maps the messages to values utilized when presenting heart rate values or other activity information during a class or other streamed content. The metrics service 215 is application scoped, because certain activities (e.g., in class and setup devices activities) will access the metrics service 215 to display metrics and state of connection on an associated dialog (e.g., SetupDevicesActivity dialog).

The data store 217 is also application scoped and stores information associated with the state of devices, such as various flow APIs. For example, the data store 217 can include APIs that enable flows that emit state updates, values, and so on to various devices. Example APIs include a MutableStateFlow API and a MutableSharedFlow API.

The metrics service 215 sends a session identifier (e.g., via a POST request) to a token authenticator 232 after a user of the peripheral device 120 provides authentication credentials. The token authenticator 232 authenticates the user via an authentication service 240, and when the user is authenticated to the service, issues a token (e.g., a JWT).

The token is then sent from the metrics service 215 to a gRPC client 234, which provides a ReceiveMetrics gRPC service, which facilitates the sending of metrics information to the peripheral device. The gRPC client 234 communicates (e.g., sends requests) with a data service 245 (e.g., a “Tangerine Service”), which provides a stream of MetricPackets to the gRPC client 234.

The gRPC client 234 utilizes a packet mapper (e.g., the MetricsPacketMapper) 236 to map the MetricPacket information into response information (e.g., TangerineResponse) to consume different sets of data (e.g., inclass-heart-rate-platform-home and companion-devices-platform-home). The consumed data is sent to the metrics service 215 and utilized by the device module 210 to display various metrics (e.g., heart rate information) to a user via a user interface of the peripheral device 120.

The architecture 200 supports various connected/disconnected states. For example, during a class (e.g., once a class has started) the system opens a connection to the gRPC stream and maintains the connection throughout the class, unless a direct connection is established between the peripheral device 120 and the exercise machine 110 (e.g., via Bluetooth or a Gymkit connection).

In addition to service timeouts, the device module 210 includes a configurable timer that uses coroutines to check for the time of the last packet. If the last packet was received outside of a configurable time window, the device module 210 or client can assume the service is disconnected and shut down by calling an API to terminate the service. During other timeouts (e.g., pausing a workout), the service can terminate and start a new stream upon the resuming of a workout or a new class.

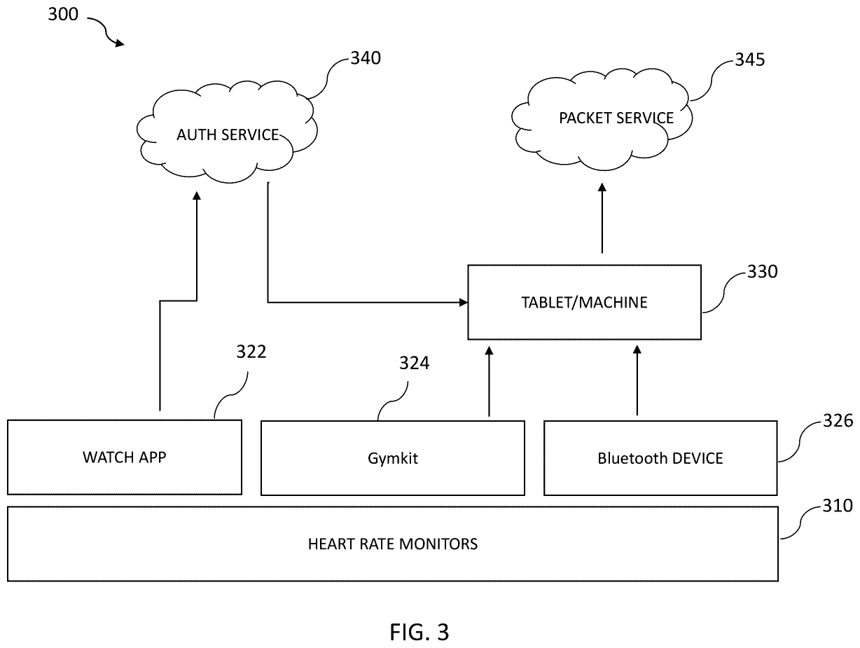

As described herein, in some embodiments, the systems and methods described herein provide functionality to peripheral devices that are capable of directly connecting to an exercise machine. FIG. 3 is a block diagram illustrating a data flow architecture 300 that provides a backward capability for a peripheral device communicating with an exercise machine.

A heart rate monitor (HRM) 310 may include one or more applications or clients 324, 326 that facilitate and/or establish a direct connection to an exercise machine, such as a tablet 330 of the machine that exchanges metrics with the HRM 310. When the HRM 310 includes a watch application 322, the application 322 can utilize a metrics service 340, as described herein, by sending heart rate information to the tablet 330 via the metrics service 340.

However, when a direct connection is established, the connection to the metrics service 340 ends, and the HRM 310 exchanges the heart rate information (and other metrics) directly to the tablet 330. The tablet can utilize a packet service 345, which receives packets having the HR information from the tablet 330.

The packet service 345, or another remote data service as described herein, in some cases, acts as an endpoint (e.g.,/packet), or API monolith, which functions to accept workout or activity metrics (e.g., heart rate, cadence, resistance, output, calories, incline, speed, and so on) generated by exercise machines during exercise activities. The packet service 345, or endpoint, manages the packets by queuing the packets for storage and updating real-time services that utilize the metrics during live class or other streamed content. For example, the packet service 345 can be a “/stats/packet” endpoint that:
Queues packets in a packet storage SQS queue;
Updates a live/here now leaderboard redistribution;
Updates a heart rate leaderboard service; and so on.

However, in some embodiments, the data service 245 of FIG. 2 can replace or augment the packet service 345 or endpoint, and function as a metrics service for peripheral devices and/or exercise machines within a connected fitness platform. Further details regarding the utilization of the data service 245 as a metrics service are described herein.

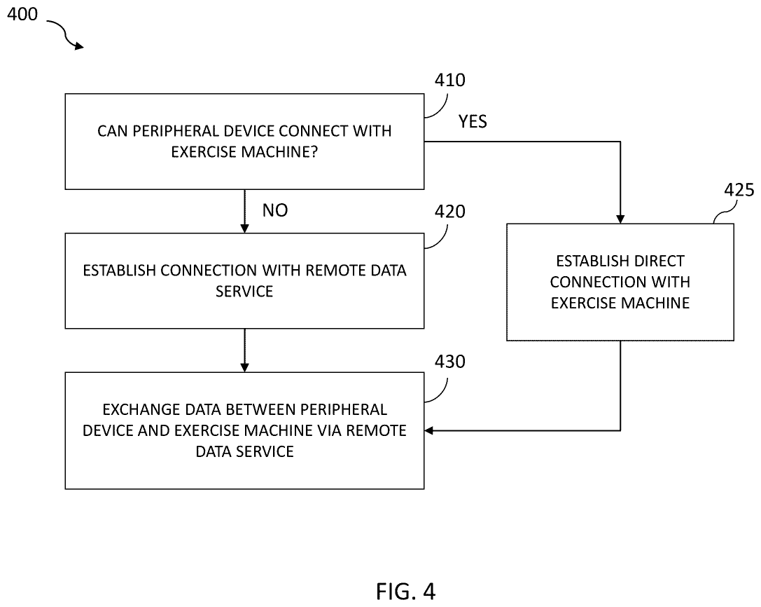

As described herein, the metrics service 215 operates to exchange metrics with devices that cannot connect to exercise machines, such as peripheral devices that are not associated with machine-specific modules or applications. FIG. 4 is a flow diagram illustrating a method 400 for transmitting data between a peripheral device and an exercise machine. The method 400 may be performed by the metrics service 215 and/or the data service 130 and, accordingly, is described herein merely by way of reference thereto. It will be appreciated that the method 400 may be performed on any suitable hardware.

In operation 410, the metrics service 215 and/or the data service 130 determines whether a peripheral device can connect with an exercise machine. If the peripheral device can directly connect with the exercise machine (e.g., via a GymKit or other similar application), the method 400 moves to operation 425, and the peripheral device directly connects with the exercise machine. If the peripheral device cannot connect directly with the exercise machine, the method 400 proceeds to operation 420.

In operation 420, the data service 130 facilitates a connection with the peripheral device. For example, the data service 130 can establish a connection with the peripheral device, such as between the metrics service 215 and the data service 130.

In operation 430, using the established connection, the peripheral device exchanges data (e.g., metrics data such as heart rate data) with the exercise machine via the data service 130. The data service 130, which can be remote to the peripheral device and/or the exercise machine, acts as an intermediary between the peripheral device and exercise machine.

As described herein, in some embodiments, the systems and methods provide and/or facilitate a service or microservice for workout metrics ingestion and coordination between devices. In some cases, the microservice can augment and/or replace use of API monoliths or other non-distributed data architectures. For example, the microservice, acting as a collection or group of services, can function to segment or decouple the different data services, such as metrics ingestion, for a connected fitness platform, as is described herein.

In some cases, the microservice can set up or provide a common message broker service for packets, such that a packet ingestion service can perform a single write operation to the broker, which fans outs messages to various services that utilize packet data.

Utilizing a microservice for packet ingestion cab realize various benefits or enhancements, including:
Better performance of the service: by moving off of a python-based api monolith, the architecture can write a more performant service that may require fewer servers per user. Longer term, clients can send metrics directly to the service using a more performant streaming protocol;
Independent scaling of services: by fanning out packet logic to each individual service, the architecture can scale independently based on service-specific computing needs;
Data Loss risk mitigation: by decoupling downstream services from the point of ingestion, the architecture can isolate potential performance issues to individual services with minimal risk to packet storage;
Less use of a monolith: the architecture can facilitate a transition from the monolith to microservices (e.g.,/stats/packet requests represent 55% of all api requests to the monolith); and so on.

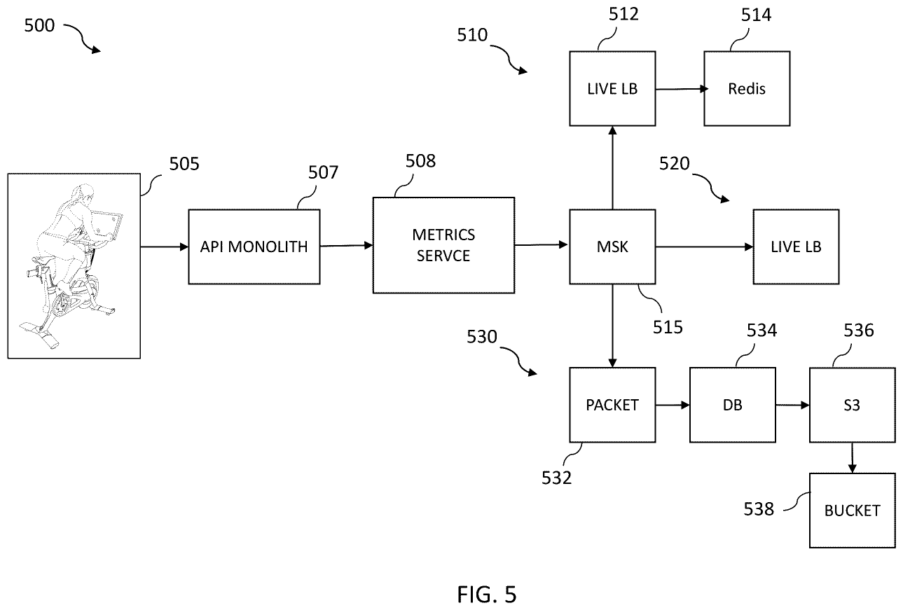

FIG. 5 is a data flow diagram 500 illustrating the flow of data within a cloud-based metrics service. As described herein, the data service 130 is configured to be a metrics service 508 that takes the place of the/stats/packet endpoint, providing a pub/sub approach to ingesting metric packets via an Amazon MSK component (Managed Kafka) 515. The metrics service 508 sends all metrics packets to a Kafka topic (e.g., MSK 515), which downstream services can then pull from. The architecture 500 allows for the decoupling and individual scaling of services that receive and/or process metric packets and allows the system to offload message broadcasting/storage to MSK while only performing one external API call to MSK from the metrics service.

For example, a packet originates from an exercise machine 505 (e.g., containing metrics and/or class data) and takes various paths from an API monolith 507 via the metrics service 508. In one path, the metrics service 508 routes the packet to a leaderboard path 510, which includes a Live Leaderboard Redis Set 514 or data structure store, which is utilized by a leaderboard application associated with the exercise machine 505.

In another path, the metrics service 508 routes the packet to a Heart Rate Leaderboard Service path 520. Further, in another path, the metrics service 508 routes the packet to a database path 530, where a packet 532 is stored in a database 534 associated with an On Demand Leaderboard, a storage service 536, and/or a Packet SQS Queue→DynamoDB bucket 538.

Thus, the system, via the metrics service 508, enables consumers to pull from the Kafka packet topic and process the packets.

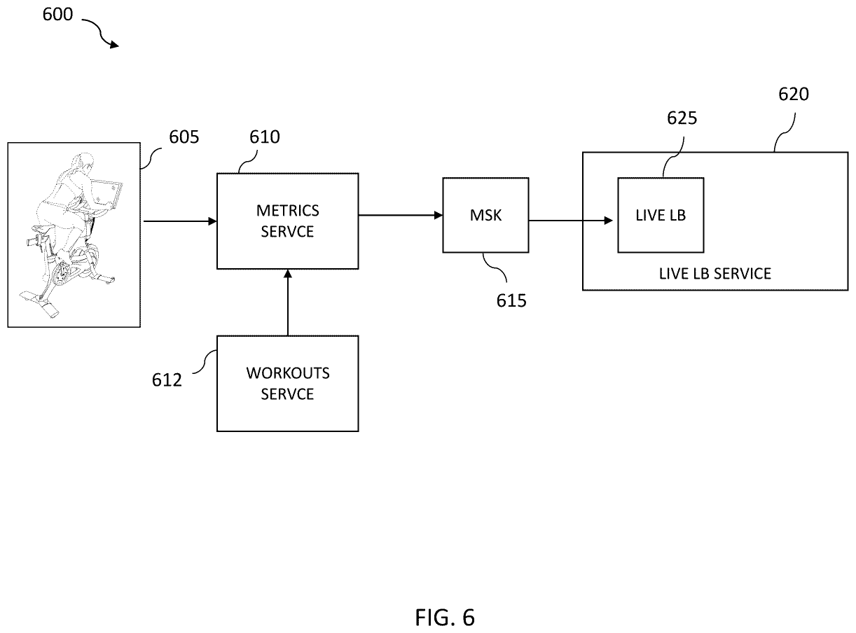

FIG. 6 is a data flow diagram 600 illustrating the flow of data within a cloud-based metrics service having a separate metrics service. The architecture, as depicted, can enable clients to directly access a metrics service 610, such as via a gPRC stream. The metrics service 610, receiving metrics and other data from an exercise machine 605, communicates with an MSK 615, which is accessible via a microservice, such as a leaderboard service 620 that manages and updates a live leaderboard for the exercise machine 605 (and other exercise machines) for classes and other content streamed by the exercise machine 605. Thus, the architecture 600 can split out the live leaderboard service 620 into its own microservice.

For example, the leaderboard service 620 can manage and/or update a leaderboard 625, such as a dynamically updated ranking of multiple users or performers of a class or other exercise activity (e.g., riders of exercise bicycles during an exercise class). The leaderboard 625 can display the relative performance of all riders, or one or more subgroups of riders. For example, the user may be able to select a leaderboard that shows the performance of riders in a particular age group, male riders, female riders, male riders in a particular age group, riders in a particular geographic area, and so on. Users may be provided with the ability to deselect the leaderboard entirely and remove it from the screen.

Further, the system can incorporate various social networking aspects, such as allowing the user to follow other riders, or to create groups or circles of riders. Thus, user lists and information may be accessed, sorted, filtered, and used in a wide range of different ways. For example, other users can be sorted, grouped and/or classified based on any characteristic including personal information such as age, gender, weight, or based on performance such as current power output, speed, or a custom score.

The leaderboard 625 can be fully interactive, allowing the user to scroll up and down through user rankings, and to select a user to access their detailed performance data, create a connection (such as choosing to follow that user), or establish direct communication (such as through an audio and/or video connection). The leaderboard 625 can also display the user's personal best performance in the same or a comparable class, to allow the user to compare their current performance to their previous personal best. The leaderboard 625 can also highlight certain users, such as those that the user follows, or provide other visual cues to indicate a connection or provide other information about a particular entry on the leaderboard.

In some cases, the leaderboard 625 can also allow the user to view their position and performance information at all times while scrolling through the leaderboard. For example, when the user scrolls up toward the top of the leaderboard 625 (such as by dragging their fingers upward on a touchscreen display presenting the leaderboard 625), when the user's window reaches the bottom of the leaderboard, it will lock in position and the rest of the leaderboard will scroll underneath it. Similarly, when the user scrolls down toward the bottom of the leaderboard, when the user's window reaches the top of the leaderboard, it will lock in position and the rest of the leaderboard will continue to scroll underneath it.

The leaderboard service 620, therefore, can facilitate the various leaderboard actions described herein based on receiving the packets/data from the metrics service 610. Further, for MSK consumers, the architecture 600 can utilize Kafka Streams and Kafka Connect to easily stream data directly from the MSK 615 to other data stores, such as Redis, DynamoDB, S3, and other described herein.

The data service 130 and associated metrics services (e.g., metrics service 215) described herein can be implemented in a variety of ways. For example, the service can be implemented in Kotlin using Micronaut with streaming gRPC and a fallback to HTTP, where useful. Otherwise, services can utilize gRPC where possible.

The MSK consumers can also be implemented as Micronaut services in k8s. Each downstream service may have its own consumer group(s), which pull messages from the Kafka topic(s) to which it is subscribed. Each downstream consumer service can be independently scaled up or down by Modifying a number of consumer/consumer groups and/or Modifying consumer pod resources.

The metrics service can fetch information about the workout_id supplied to the stream. The fetching of information validates that the workout belongs to the user uploading the metrics. The service can also fetch data about the workout to tag information about workout eligibility on the live leaderboard or for other services. The metrics service can call an external service that can return workout data, such as by making a call to the api monolith or by creating a new workout service. In either case, the workout data can be cached for the duration of a gRPC stream or workout, so the added load should be minimal.

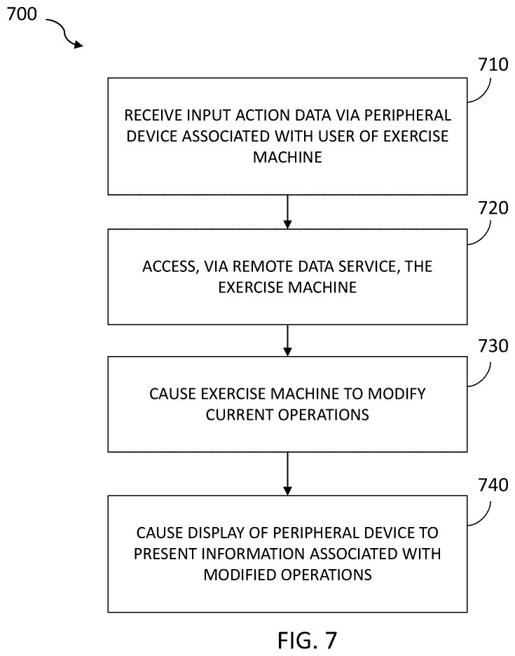

As described herein, use of the systems and methods described herein can facilitate the peripheral device as a second user interface for an exercise class presented by an exercise machine. FIG. 7 is a flow diagram illustrating a method 700 for controlling an exercise machine with a peripheral device via a cloud-based metrics service. The method 700 may be performed by the data service 130 and, accordingly, is described herein merely by way of reference thereto. It will be appreciated that the method 700 may be performed on any suitable hardware.

In operation 710, the peripheral device receives input action data from a user performing a workout using an exercise machine. For example, the peripheral device can receive user input via a touch screen, via voice commands, via one or more controls, and so on.

In operation 720, the data service 130 accesses the exercise machine. As described herein, the peripheral device can be connected to the data service 130, which receives the input data from the peripheral device.

In operation 730, the data service 130 causes the exercise machine to modify current operations (e.g., display different information, change machine settings, and so on).

In operation 740, the data service 130 causes a display of the peripheral device to present information associated with the modified operations. For example, the peripheral device, acting as a second screen for the class or workout, updates the display to show the changed machine settings.

Thus, the data service 130, in some cases, can function to facilitate the modification of the operation of the exercise machine, such as during an exercise activity performed by a user during a streamed exercise class.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the technology may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

What is claimed is:

1. A system of a peripheral device that is associated with an exercise machine, the system comprising:
a processor; and
memory connected with the processor, wherein the memory stores instructions that cause the processor to perform a method, the method including:
determining whether the peripheral device can establish a direct connection with the exercise machine; and
when the peripheral device cannot establish the direct connection with the exercise machine:
establishing a connection with a remote data service associated with the exercise machine; and
exchanging data captured from a user of the exercise machine with the exercise machine via the established connection with the remote data service.

2. The system of claim 1, further comprising:
when the peripheral device can establish the direct connection with the exercise machine:
establishing the direct connection with the exercise machine; and
exchanging data captured from the user of the exercise machine with the exercise machine via the direct connection.

3. The system of claim 1, wherein the data captured from the user of the exercise machine includes heart rate data captured during an exercise activity performed by the user on the exercise machine.

4. The system of claim 1, wherein the peripheral device is a smart watch worn by the user during an exercise activity performed by the user on the exercise machine.

5. The system of claim 1, wherein the peripheral device is a heart rate monitor worn by the user during an exercise activity performed by the user on the exercise machine.

6. The system of claim 1, wherein the exercise machine is an exercise bicycle, and wherein the remote data service is located at a server remote from the exercise bicycle that is configured to stream content displayed by the exercise bicycle during an exercise activity performed by the user on the exercise machine.

7. The system of claim 1, wherein the exercise machine is a treadmill, and wherein the remote data service is located at a server remote from the treadmill that is configured to stream content displayed by the exercise bicycle during an exercise activity performed by the user on the exercise machine.

8. The system of claim 1, wherein establishing a connection with a remote data service associated with the exercise machine includes establishing a connection between a metrics service of the peripheral device and the remote data service.

9. The system of claim 1, wherein the system is part of a mobile application supported by the peripheral device.

10. A method performed by a peripheral device associated with an exercise machine, the method comprising:
determining whether the peripheral device can establish a direct connection with the exercise machine; and
when the peripheral device cannot establish the direct connection with the exercise machine:
establishing a connection with a remote data service associated with the exercise machine; and
exchanging data captured from a user of the exercise machine with the exercise machine via the established connection with the remote data service.

11. The method of claim 10, further comprising:
when the peripheral device can establish the direct connection with the exercise machine:
establishing the direct connection with the exercise machine; and
exchanging data captured from the user of the exercise machine with the exercise machine via the direct connection.

12. The method of claim 10, wherein the data captured from the user of the exercise machine includes heart rate data captured during an exercise activity performed by the user on the exercise machine.

13. The method of claim 10, wherein the peripheral device is a smart watch worn by the user during an exercise activity performed by the user on the exercise machine.

14. The method of claim 10, wherein the peripheral device is a heart rate monitor worn by the user during an exercise activity performed by the user on the exercise machine.

15. The method of claim 10, wherein the exercise machine is an exercise bicycle, and wherein the remote data service is located at a server remote from the exercise bicycle that is configured to stream content displayed by the exercise bicycle during an exercise activity performed by the user on the exercise machine.

16. The method of claim 10, wherein the exercise machine is a treadmill, and wherein the remote data service is located at a server remote from the treadmill that is configured to stream content displayed by the exercise bicycle during an exercise activity performed by the user on the exercise machine.

17. The method of claim 10, wherein establishing a connection with a remote data service associated with the exercise machine includes establishing a connection between a metrics service of the peripheral device and the remote data service.

18. The method of claim 10, wherein the system is part of a mobile application supported by the peripheral device.

19. A connected fitness platform, comprising:
multiple exercise machines that are configured to capture data from users performing exercise activities during streamed classes;
a metrics service that receives data packets, from the multiple exercise machines, containing the captured data; and
one or more microservices that access the metrics service to update an interactive service provided to the multiple exercise machines by the connected fitness platform.

20. The connected fitness platform of claim 19, wherein the one or more microservices include a leaderboard service that presents a leaderboard of users of the multiple exercise machines during a specific streamed class.
